(19) United States
(12) Patent Application Publication
(10) Pub. No.: US 2024/0216669 A1
(43) Pub. Date: Jul. 4, 2024

(54) BLOOD CIRCULATION SYSTEM

(71) Applicant: TERUMO KABUSHIKI KAISHA, TOKYO (JP)

(72) Inventor: Takehiko Mori, Kanagawa (JP)

(21) Appl. No.: 18/609,490

(22) Filed: Mar. 19, 2024

Related U.S. Application Data
(63) Continuation of application No. PCT/JP2022/034867, filed on Sep. 20, 2022.

(30) Foreign Application Priority Data
Sep. 27, 2021 (JP) 2021-156478

(51) Int. Cl. A61M 60/117; A61M 60/113; A61M 60/232; A61M 60/38; A61M 60/419; A61M 60/523; A61M 60/585

(52) U.S. Cl. CPC A61M 60/117; A61M 60/113; A61M 60/232; A61M 60/38; A61M 60/419; A61M 60/523; A61M 60/585; A61M 2202/0021; A61M 2202/0413; A61M 2205/103; A61M 2205/3303; A61M 2205/3327; A61M 2205/3334; A61M 2205/3365; A61M 2205/505; A61M 2210/125; A61M 2210/127

(57) ABSTRACT

In a blood circulation system (10), a first blood circulation device (12) includes a first detection unit (22) that detects a first flow rate of blood flowing through a first blood sending vessel portion (28). A second blood circulation device (14) includes a second detection unit (95) that detects a second flow rate of blood flowing through a blood sending cannula (110). A first control section (74) controls a first centrifugal pump (20) based on a detection result of the second detection unit (95). A second control section (154) controls a second centrifugal pump (92) based on a detection result of the first detection unit (22).

### CROSS REFERENCE TO RELATED APPLICATIONS

This application is a continuation of PCT Application No. PCT/JP2022/034867, filed Sep. 20, 2022, based on and claiming priority to Japanese Application No. JP2021-156478, filed Sep. 27, 2021, both of which are incorporated herein by reference in their entirety.

### BACKGROUND OF THE INVENTION

The present invention relates to a blood circulation system.

Prashant Rao, Zain Khalpey, Richard Smith, Daniel Burkhoff, Robb D. Kociol, Venoarterial Extracorporeal Membrane Oxygenation for Cardiogenic Shock and Cardiac Arrest Cardinal Considerations for Initiation and Management [Circ Heart Fail. 2018; 11:e004905. DOI: 10.1161/CIRCHEARTFAILURE.118.004905] discloses a blood circulation system including a first blood circulation device and a second blood circulation device.

One type of blood circulation device is called Impella®, available from Abiomed of Danvers, Massachusetts, which is placed percutaneously to assist the function of the heart of a patient. That is, the first type of blood circulation device is a catheter inserted into the femoral artery. A distal end of the catheter is inserted into the left ventricle via the femoral artery and the ascending aorta. A first opening is formed at the distal end of the catheter. A second opening is formed at a position of the ascending aorta in the catheter. An axial flow pump is provided in the catheter between the first opening and the second opening. The axial flow pump removes blood from the left ventricle via the first opening and delivers the blood to the ascending aorta via the second opening.

A second type of blood circulation device is called “V-A ECMO” (venoarterial extracorporeal membrane oxygenation) which substitutes for the functions of the heart and lungs of the patient. That is, the second type of blood circulation device includes a blood circulation circuit, a centrifugal pump, and an oxygenator. The centrifugal pump and the oxygenator are disposed in the blood circulation circuit. The centrifugal pump aspirates blood from the patient's femoral vein and sends it to the oxygenator. The oxygenator oxygenates the blood. The oxygenated blood is delivered to the femoral artery of the patient.

In a case where only the second type of blood circulation device is used, the burden on the patient's heart increases. On the other hand, when the first type of blood circulation device and the second blood circulation device are used in combination, the burden on the heart of the patient is reduced, and a survival rate of the patient is improved.

The condition of the patient changes over time. In a case where the first type of blood circulation device and the second type of blood circulation device are used in combination, it is necessary to appropriately adjust the flow rates of blood in the first blood circulation device and the second blood circulation device according to the condition of the patient. Note that the flow rate of blood in the first blood circulation device is an amount of blood (blood removal amount) sucked from the left ventricle through the first opening. The blood removal amount is also referred to as an unloading flow rate. The unloading flow rate is also an amount of blood (a blood sending amount) sent from the axial flow pump to the ascending aorta via the second opening.

However, in the first (percutaneous) blood circulation device, since the axial flow pump is built in the catheter, the flow rate (unloading flow rate) of the axial flow pump cannot be directly measured. Furthermore, the axial flow pump has a pump characteristic in which the unloading flow rate sharply changes due to a pressure difference between an inlet and an outlet of the axial flow pump. Therefore, the unloading flow rate cannot be accurately calculated based on a rotation speed of the axial flow pump and a current flowing through a motor that drives the axial flow pump. As a result, the axial flow pump cannot be appropriately controlled based on the calculated unloading flow rate. Therefore, the unloading flow rate cannot be appropriately adjusted according to the condition of the patient.

Furthermore, also in the second blood circulation device, it is necessary to appropriately control the blood sending amount to the patient. However, since the first blood circulation device and the second blood circulation device are used in combination, it is difficult to appropriately control the blood sending amount. As a result, the burden on the medical worker increases.

### SUMMARY OF THE INVENTION

An object of the present invention is to provide a blood circulation system capable of appropriately controlling a flow rate of blood according to the condition of a patient.

A first aspect of the present invention is a blood circulation system that removes blood from a living body and sends the blood to the living body, the blood circulation system including: a first blood circulation device; and a second blood circulation device, wherein the first blood circulation device includes: a first blood removal vessel portion having a distal end inserted into a left ventricle of the living body via a subclavian artery of the living body; a first blood sending vessel portion having a distal end inserted into an ascending aorta of the living body via the subclavian artery; a first centrifugal pump that is disposed outside the living body, includes a first inlet port and a first outlet port, removes blood from the left ventricle to the first inlet port via the first blood removal vessel portion, and sends blood from the first outlet port to the ascending aorta via the first blood sending vessel portion, the first inlet port being connected to a proximal end of the first blood removal vessel portion, the first outlet port being connected to a proximal end of the first blood sending vessel portion; and a first control section that controls the first centrifugal pump, and wherein the second blood circulation device includes: a second blood removal vessel portion having a distal end inserted into a femoral vein of the living body; a second blood sending vessel portion having a distal end inserted into a femoral artery of the living body; a second centrifugal pump that is disposed outside the living body, includes a second inlet port and a second outlet port, removes blood from the femoral vein to the second inlet port via the second blood removal vessel portion, and sends blood from the second outlet port to the femoral artery via the second blood sending vessel portion, the second inlet port being connected to a proximal end of the second blood removal vessel portion, the second outlet port being connected to a proximal end of the second blood sending vessel portion; a second control section that controls the second centrifugal pump; and a detection unit that is disposed outside the living body and detects a flow rate of blood flowing through the second blood sending vessel portion, and the first control section controls the first centrifugal pump based on a detection result of the detection unit that detects the flow rate of blood flowing from the second centrifugal pump through the second blood sending vessel portion.

According to the present invention, the centrifugal pump of one of the blood circulation devices can be controlled based on the detection result of a flow rate detection unit provided in the other one of the blood circulation devices. As a result, the flow rate of the blood flowing through each blood circulation device can be appropriately controlled according to the condition of a patient.

In the above blood circulation system, it is preferable that the first blood circulation device further includes a flow rate calculation unit that calculates a flow rate of blood flowing into the first blood sending vessel portion from a target flow rate flowing from the first blood circulation device and the second blood circulation device to the ascending aorta and a detection result of the detection unit, and the first control section controls a rotation speed of the first centrifugal pump based on the flow rate calculated by the flow rate calculation unit.

This makes it possible to accurately control the flow rate of the blood flowing through the other blood circulation device while considering the state of one blood circulation device.

A second aspect of the present invention is a blood circulation system that removes blood from a living body and sends the blood to the living body, the blood circulation system including: a first blood circulation device and a second blood circulation device, wherein the first blood circulation device includes: a first blood removal vessel portion having a distal end inserted into a left ventricle of the living body via a subclavian artery of the living body; a first blood sending vessel portion having a distal end inserted into an ascending aorta of the living body via the subclavian artery; a first centrifugal pump that is disposed outside the living body, includes a first inlet port and a first outlet port, removes blood from the left ventricle to the first inlet port via the first blood removal vessel portion, and sends blood from the first outlet port to the ascending aorta via the first blood sending vessel portion, the first inlet port being connected to a proximal end of the first blood removal vessel portion, the first outlet port being connected to a proximal end of the first blood sending vessel portion; a first control section that controls the first centrifugal pump; and a detection unit that is disposed outside the living body and detects a flow rate of blood flowing through the first blood sending vessel portion, wherein the second blood circulation device includes: a second blood removal vessel portion having a distal end inserted into a femoral vein of the living body; a second blood sending vessel portion having a distal end inserted into a femoral artery of the living body; a second centrifugal pump that is disposed outside the living body, includes a second inlet port and a second outlet port, removes blood from the femoral vein to the second inlet port via the second blood removal vessel portion, and sends blood from the second outlet port to the femoral artery via the second blood sending vessel portion, the second inlet port being connected to a proximal end of the second blood removal vessel portion, the second outlet port being connected to a proximal end of the second blood sending vessel portion; and a second control section that controls the second centrifugal pump; wherein the second control section controls the second centrifugal pump based on a detection result of the detection unit which detects the flow rate of blood flowing from the first centrifugal pump through the first blood sending vessel portion.

According to the present invention, the centrifugal pump of one of the blood circulation devices can be controlled based on the detection result of the detection unit provided in the other one of blood circulation devices. As a result, the flow rate of the blood flowing through each blood circulation device can be appropriately controlled according to the condition of a patient.

In the blood circulation system, it is preferable that the second blood circulation device further includes a flow rate calculation unit that calculates a flow rate of blood flowing into the second blood sending vessel portion from a target flow rate flowing from the first blood circulation device and the second blood circulation device to the ascending aorta and a detection result of the detection unit, and the second control section controls a rotation speed of the second centrifugal pump based on the flow rate calculated by the flow rate calculation unit.

This makes it possible to accurately control the flow rate of the blood flowing through the other blood circulation device while considering the state of one blood circulation device.

### BRIEF DESCRIPTION OF THE DRAWINGS

FIG. 1 is a configuration diagram of a blood circulation system according to the present embodiment.

FIG. 2 is an explanatory diagram of a heart illustrating a state in which a distal end of a cannula is inserted into a left ventricle.

FIG. 3 is a diagram illustrating pump characteristics of a first centrifugal pump and a second centrifugal pump.

FIG. 4 is an explanatory diagram of the vicinity of Valsalva sinus.

FIG. 5 is a perspective view of a heart.

### DETAILED DESCRIPTION OF PREFERRED EMBODIMENTS

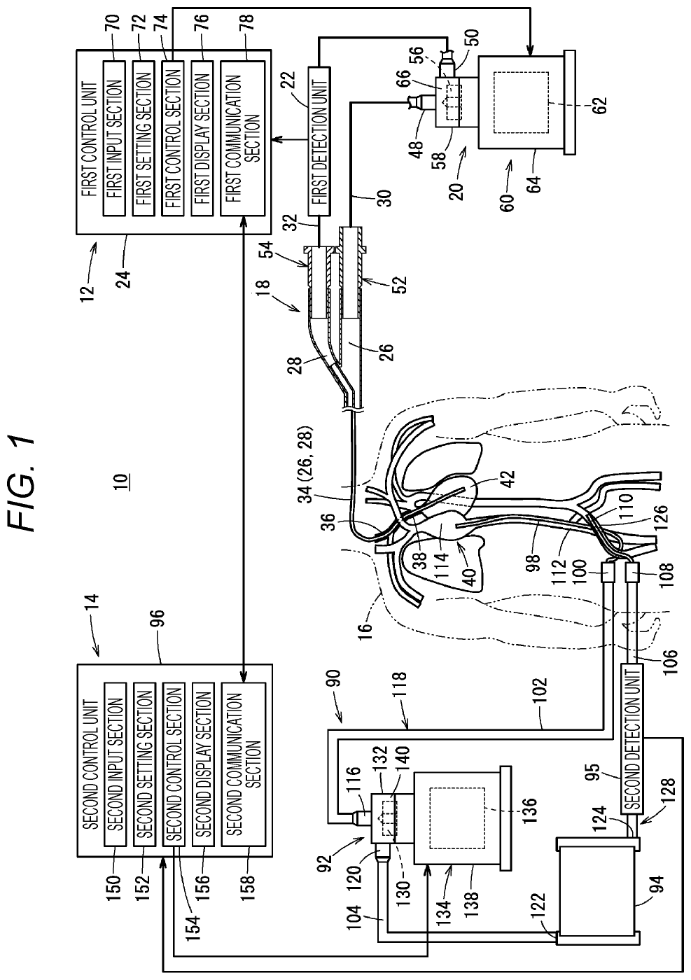

As illustrated in FIG. 1, a blood circulation system 10 according to the present embodiment includes a first blood circulation device 12 and a second blood circulation device 14.

The first blood circulation device 12 includes a first blood circulation circuit 18 that removes blood from a patient 16 (living body) and sends the blood to the patient 16. The first blood circulation circuit 18 forms a path for extracorporeally circulating blood by sucking blood from the patient 16 and returning the blood to the patient 16 again. The first blood circulation device 12 further includes a first centrifugal pump 20, a first detection unit 22, and a first control unit 24. The first centrifugal pump 20 is disposed in the first blood circulation circuit 18. The first centrifugal pump 20 assists a heart function of the patient 16. The first blood circulation device 12 circulates the blood of the first blood circulation circuit 18 by driving the first centrifugal pump 20.

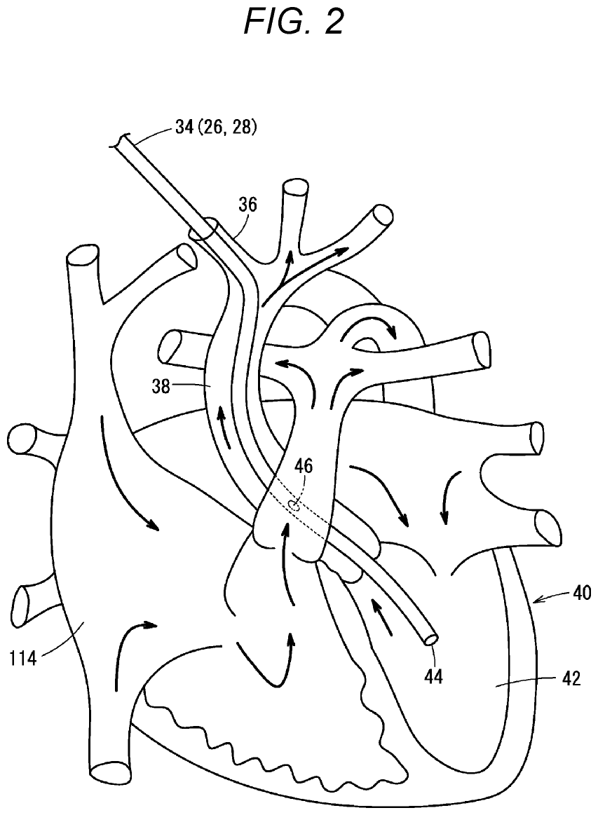

The first blood circulation circuit 18 includes a first blood removal vessel portion 26, a first blood sending vessel portion 28, a first blood removal tube 30, and a first blood sending tube 32. In the first blood circulation circuit 18, the first blood removal vessel portion 26, the first blood removal tube 30, the first blood sending tube 32, and the first blood sending vessel portion 28 are disposed in this order along a direction in which blood circulates. The first blood removal vessel portion 26 and the first blood sending vessel portion 28 constitute one cannula 34 illustrated in FIGS. 1 and 2. That is, the cannula 34 is a tubular body having two hollow portions. One of the two hollow portions serves as the first blood removal vessel portion 26, and the other hollow portion serves as the first blood sending vessel portion 28.

A distal end of the cannula 34 is inserted into a left ventricle 42 of a heart 40 via a subclavian artery 36 and an ascending aorta 38 of the patient 16. A first opening 44 communicating with the first blood removal vessel portion 26 is formed at the distal end of the cannula 34. That is, a distal end of the first blood removal vessel portion 26 is inserted into the left ventricle 42. A second opening 46 communicating with the first blood sending vessel portion 28 is formed at a position of the ascending aorta 38 in the cannula 34. That is, the distal end of the first blood removal vessel portion 26 is inserted into the ascending aorta 38. Note that an arrow in FIG. 2 indicates the flow of blood in a case where the heart 40 of the patient 16 is normal.

As illustrated in FIG. 1, a proximal end of the cannula 34 is bifurcated into two portions. One branch portion is a proximal end of the first blood removal vessel portion 26. The proximal end of the first blood removal vessel portion 26 is connected to a first inlet port 48 of the first centrifugal pump 20 via the first blood removal tube 30. The other branch portion is a proximal end of the first blood sending vessel portion 28. The proximal end of the first blood sending vessel portion 28 is connected to a first outlet port 50 of the first centrifugal pump 20 via the first blood sending tube 32. Note that, in the present embodiment, as illustrated in FIG. 1, a type in which the proximal end of the cannula 34 is branched into two portions is illustrated. In the present embodiment, it is also possible to use two independent cannulae.

The first blood removal vessel portion 26, the first blood removal tube 30, and the first inlet port 48 constitute a first blood removal line 52 for removing blood from the patient 16. The first outlet port 50, the first blood sending tube 32, and the first blood sending vessel portion 28 constitute a first blood sending line 54 for sending blood to the patient 16.

The first centrifugal pump 20 functions as a power source for removing blood from the patient 16 to the outside of the body. Furthermore, the first centrifugal pump 20 functions as a power source for sending blood into the body of the patient 16. The first centrifugal pump 20 includes a first impeller 56 and a first pump housing 58 that accommodates the first impeller 56. The first centrifugal pump 20 causes the blood to flow by a centrifugal force accompanying the rotation of the first impeller 56. That is, the first centrifugal pump 20 aspirates the blood of the patient 16 via the first blood removal line 52 by negative pressure. Furthermore, the first centrifugal pump 20 sends blood to the patient 16 through the first blood sending line 54. The first pump housing 58 is formed of a resin material or the like. The first pump housing 58 is a hollow cylinder.

The first centrifugal pump 20 is detachably connected to a first drive device 60. The first drive device 60 includes a first motor 62 and a first motor housing 64 that accommodates the first motor 62. The first pump housing 58 and the first motor housing 64 are detachably connected.

The first impeller 56 is disposed in an internal space of the first pump housing 58. The first impeller 56 and the first motor 62 are magnetically coupled via a magnetic coupling portion (not illustrated). Therefore, the first impeller 56 is rotated by the drive of the first motor 62.

The first inlet port 48 is provided in the first pump housing 58. A hollow portion (flow path) of the first inlet port 48 communicates with the internal space of the first pump housing 58. The first inlet port 48 is formed at a position opposite to a position where the first motor housing 64 is mounted in the first pump housing 58. The first inlet port 48 is provided on an axis of the first impeller 56. The first inlet port 48 protrudes from the first pump housing 58 in an axial direction of the first impeller 56.

The first pump housing 58 is further provided with the first outlet port 50 communicating with the internal space of the first pump housing 58. The first outlet port 50 protrudes from a side wall 66 of the first pump housing 58 in a direction intersecting the axis of the first impeller 56.

The first centrifugal pump 20 maybe a volute pump. Furthermore, the first centrifugal pump 20 maybe an open-type pump. In a case where the first centrifugal pump 20 is an open-type pump, the first impeller 56 is not provided with a cover that covers a plurality of blades.

The first detection unit 22 is disposed outside the body of the patient 16. Specifically, the first detection unit 22 is disposed in the middle of the first blood sending tube 32. The first detection unit 22 is a flow rate sensor that detects a flow rate (first flow rate) of blood flowing through the first blood sending line 54. The first detection unit 22 successively detects the first flow rate and outputs the detection results to the first control unit 24.

The first control unit 24 includes a first input section 70, a first setting section 72 (flow rate calculation unit), a first control section 74, a first display section 76, and a first communication section 78. The first control unit 24 is a controller including the first display section 76 that is a display. The first control unit 24 controls the first blood circulation device 12 as a whole by reading and executing a program stored in a memory (not illustrated).

The first input section 70 is an operator operable by a user. The first input section 70 is, for example, a touch panel. A user such as a medical worker operates the first input section 70 to input a target flow rate for the blood circulation system 10 to flow into the ascending aorta 38. The target flow rate is also a flow rate of blood required by the patient 16.

The first setting section 72 sets a target value (first target value) of the first flow rate based on the target flow rate or the like. For example, in the blood circulation system 10, in a case where only the first blood circulation device 12 operates, the first setting section 72 sets the target flow rate to the first target value. Note that the target flow rate may change with the lapse of time according to the condition of the patient 16. Furthermore, the first target value may change with the lapse of time according to the condition of the patient 16.

The first control section 74 controls a rotation speed of the first motor 62 based on the first target value set by the first setting section 72.

Here, setting processing in the first setting section 72 and control processing of the rotation speed of the first motor 62 in the first control section 74 will be described in detail.

In order to remove blood from the left ventricle 42 and send the blood to the ascending aorta 38, it is necessary to adjust a pressure difference between the left ventricle 42 and the ascending aorta 38 to an appropriate value. Specifically, the pressure of the ascending aorta 38 needs to be higher than the pressure of the left ventricle 42.

Therefore, the first control section 74 appropriately adjusts the pressure difference between the left ventricle 42 and the ascending aorta 38 by controlling the rotation speed of the first centrifugal pump 20. Note that a rotation speed N of the first centrifugal pump 20 is also a rotation speed of the first impeller 56.

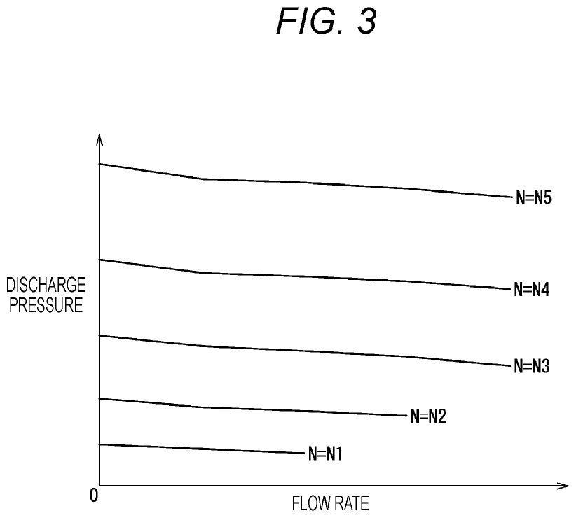

FIG. 3 illustrates pump characteristics of the first centrifugal pump 20 (see FIG. 1). The first centrifugal pump 20 has pump characteristics in which a discharge pressure is substantially constant with respect to an arbitrary rotation speed N. That is, the discharge pressure hardly depends on the first flow rate. That is, when the rotation speed N of the first centrifugal pump 20 is determined, the pressure of the blood sent to the ascending aorta 38 is determined. In other words, by referring to the pump characteristics in FIG. 3, the rotation speed N according to the pressure of the blood sent to the ascending aorta 38 can be determined. Therefore, the pressure difference between the left ventricle 42 and the ascending aorta 38 can be appropriately adjusted by changing the rotation speed N of the first centrifugal pump 20.

Note that FIG. 3 illustrates pump characteristics when the rotation speed N of the first centrifugal pump 20 is N1 to N5 (N1<N2<N3<N4<N5).

On the other hand, a pressure loss of the cannula 34 depends on the flow rate of blood. Therefore, when inner diameters of the first blood removal vessel portion 26 and the first blood sending vessel portion 28 decrease, dependency of the pressure loss on the flow rate increases. That is, the pressure loss of the cannula 34 changes according to the flow rate of blood. In other words, the flow rate of the blood passing through the cannula 34 changes according to the pressure of the blood before and after along a blood circulation direction in the cannula 34.

As illustrated in FIG. 1, the first setting section 72 includes two maps (first map, second map). The first map is a map (not illustrated) indicating a relationship between the first flow rate in the cannula 34 and a first pressure (pressure of blood flowing through the first blood sending line 54). The first setting section 72 refers to the first map to convert the first target value into the first pressure.

The second map is a map of the pump characteristics of FIG. 3. The first setting section 72 refers to the second map to determine the rotation speed N according to the converted first pressure. The first control section 74 controls the rotation speed of the first impeller 56 by controlling the rotation speed of the first motor 62 based on the determined rotation speed N. As a result, the first flow rate and the first pressure are controlled, and the pressure difference between the left ventricle 42 and the ascending aorta 38 can be appropriately adjusted.

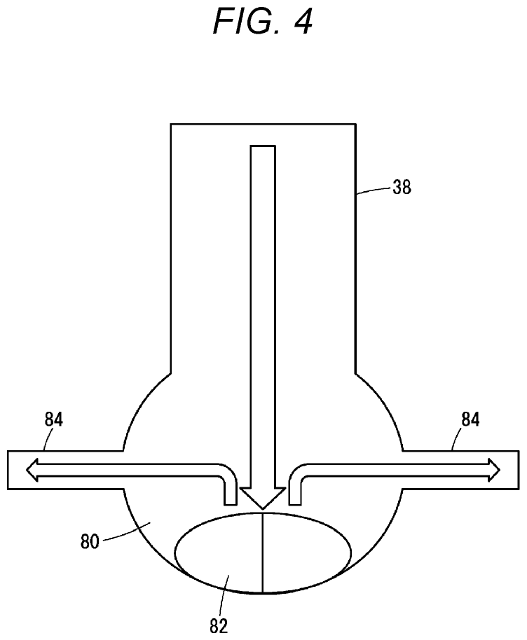

By appropriately adjusting the pressure difference between the left ventricle 42 and the ascending aorta 38, a part of the blood sent to the ascending aorta 38 flows toward a sinus of Valsalva 80 as illustrated in FIG. 4.

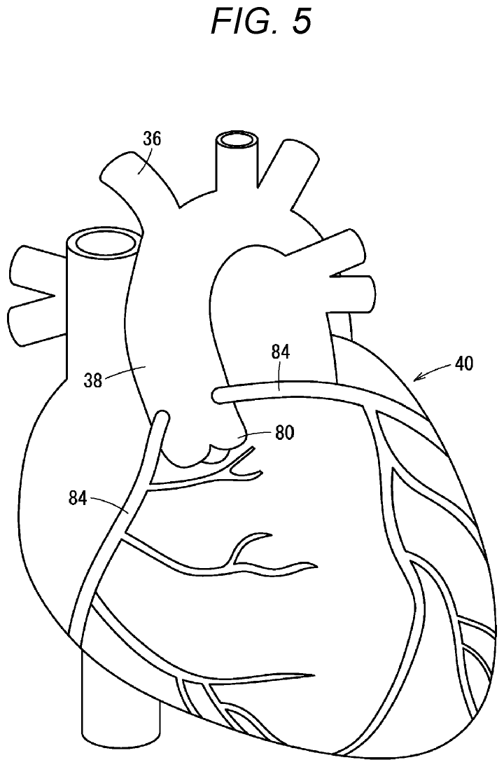

Due to this pressure difference, an aortic valve 82 formed at a proximal end of the ascending aorta 38 is closed. When the aortic valve 82 is closed, as illustrated in FIGS. 4 and 5, the blood flowing into the sinus of Valsalva 80 flows from the sinus of Valsalva 80 to left and right coronary arteries 84. As a result, it is possible to suppress the occurrence of thrombus in the sinus of Valsalva 80. Furthermore, the blood sending to the left and right coronary arteries 84 can be maintained.

Note that the detection result of the first detection unit 22 is sequentially input to the first control unit 24 (see FIG. 1). Therefore, the first setting section 72 and the first control section 74 may perform feedback control on the first flow rate on the basis of the first target value and the detection result of the first detection unit 22.

The first display section 76 displays various display contents. The first communication section 78 transmits and receives signals or information to and from a second control unit 96.

As illustrated in FIG. 1, the second blood circulation device 14 includes a second blood circulation circuit 90 that removes blood from the patient 16 and sends the blood to the patient 16. The second blood circulation circuit 90 forms a path for extracorporeally circulating blood by sucking blood from the patient 16 and returning the blood to the patient 16 again. The second blood circulation device 14 is a device called “V-A ECMO” that removes blood from a vein of the patient 16 and sends the blood to an artery of the patient 16. The second blood circulation device 14 further includes a second centrifugal pump 92, an oxygenator 94, a second detection unit 95, and the second control unit 96. The second centrifugal pump 92 is disposed in the second blood circulation circuit 90. The second centrifugal pump 92 assists the heart function of the patient 16. The oxygenator 94 is disposed in the second blood circulation circuit 90. The oxygenator 94 is an extracorporeal membrane oxygenator and assists a pulmonary function of the patient 16. The second blood circulation device 14 circulates the blood of the second blood circulation circuit 90 by driving the second centrifugal pump 92. Note that the second centrifugal pump 92 may have the same pump characteristics as those of the first centrifugal pump 20 (see FIG. 3).

The second blood circulation circuit 90 includes a blood removal cannula 98 (second blood removal vessel portion), a first connector 100, a second blood removal tube 102, a second blood sending tube 104, a third blood sending tube 106, a second connector 108, and a blood sending cannula 110 (second blood sending vessel portion). In the second blood circulation circuit 90, the blood removal cannula 98, the first connector 100, the second blood removal tube 102, the second blood sending tube 104, the third blood sending tube 106, the second connector 108, and the blood sending cannula 110 are disposed in this order along a direction in which blood circulates.

The blood removal cannula 98 is inserted into a femoral vein 112, for example. A distal end of the blood removal cannula 98 is inserted into a right atrium 114 of the heart 40 of the patient 16 via the femoral vein 112. A proximal end of the blood removal cannula 98 is connected to the first connector 100 outside the body of the patient 16. One end of the second blood removal tube 102 is connected to the first connector 100. The other end of the second blood removal tube 102 is connected to a second inlet port 116 of the second centrifugal pump 92. The blood removal cannula 98, the first connector 100, the second blood removal tube 102, and the second inlet port 116 constitute a second blood removal line 118 for removing blood from the patient 16.

One end of the second blood sending tube 104 is connected to a second outlet port 120 of the second centrifugal pump 92. The other end of the second blood sending tube 104 is connected to an inlet port 122 of the oxygenator 94. One end of the third blood sending tube 106 is connected to an outlet port 124 of the oxygenator 94. The other end of the third blood sending tube 106 is connected to the second connector 108 outside the body of the patient 16. A proximal end of the blood sending cannula 110 is connected to the second connector 108. The blood sending cannula 110 is inserted into a femoral artery 126, for example. Therefore, a distal end of the blood sending cannula 110 is inserted into the femoral artery 126. The second outlet port 120, the second blood sending tube 104, the oxygenator 94, the third blood sending tube 106, the second connector 108, and the blood sending cannula 110 constitute a second blood sending line 128 for sending blood to the patient 16.

The second centrifugal pump 92 functions as a power source for removing blood from the patient 16 to the outside of the body. Furthermore, the second centrifugal pump 92 functions as a power source for sending blood into the body of the patient 16. The second centrifugal pump 92 has substantially the same configuration as the first centrifugal pump 20. That is, the second centrifugal pump 92 includes a second impeller 130 and a second pump housing 132 that accommodates the second impeller 130. The second centrifugal pump 92 causes the blood to flow by a centrifugal force accompanying the rotation of the second impeller 130. That is, the second centrifugal pump 92 aspirates the blood of the patient 16 via the second blood removal line 118 by negative pressure.

Furthermore, the second centrifugal pump 92 sends blood to the patient 16 through the second blood sending line 128. The second pump housing 132 is formed of a resin material or the like. The second pump housing 132 is a hollow cylinder.

The second centrifugal pump 92 is detachably connected to a second drive device 134. The second drive device 134 includes a second motor 136 and a second motor housing 138 that accommodates the second motor 136. The second pump housing 132 and the second motor housing 138 are detachably connected.

The second impeller 130 is disposed in an internal space of the second pump housing 132. The second impeller 130 and the second motor 136 are magnetically coupled via a magnetic coupling portion (not illustrated). Therefore, the second impeller 130 is rotated by the drive of the second motor 136.

The second inlet port 116 is provided in the second pump housing 132. A hollow portion (flow path) of the second inlet port 116 communicates with an internal space of the second motor housing 138. The second inlet port 116 is formed at a position opposite to a position where the second motor housing 138 is mounted in the second pump housing 132. The second inlet port 116 is provided on an axis of the second impeller 130. The second inlet port 116 protrudes from the second pump housing 132 in an axial direction of the second impeller 130.

The second pump housing 132 is further provided with the second outlet port 120 communicating with the internal space of the second pump housing 132. The second outlet port 120 protrudes from a side wall 140 of the second pump housing 132 in a direction intersecting the axis of the second impeller 130.

The second centrifugal pump 92 maybe a volute pump. Furthermore, the second centrifugal pump 92 may be an open-type pump. In a case where the second centrifugal pump 92 is an open-type pump, the second impeller 130 is not provided with a cover that covers a plurality of blades.

The second detection unit 95 is disposed outside the body of the patient 16. Specifically, the second detection unit 95 is disposed in the middle of the third blood sending tube 106. The second detection unit 95 is a flow rate sensor that detects a flow rate (second flow rate) of blood flowing through the second blood sending line 128. The second detection unit 95 sequentially detects the second flow rate and outputs the detection result to the second control unit 96.

The second control unit 96 includes a second input section 150, a second setting section 152 (flow rate calculation unit), a second control section 154, a second display section 156, and a second communication section 158. The second control unit 96 is a controller including the second display section 156 that is a display. The second control unit 96 controls the second blood circulation device 14 as a whole by reading and executing a program stored in a memory (not illustrated).

The second input section 150 is an operator operable by a user. The second input section 150 is, for example, a touch panel. A user such as a medical worker operates the second input section 150 to input a target flow rate.

The second setting section 152 calculates a target value (second target value) of the second flow rate based on the target flow rate and the like. For example, in the blood circulation system 10, in a case where only the second blood circulation device 14 operates, the second setting section 152 sets the target flow rate to the second target value. Note that, as described above, the target flow rate may change with the lapse of time according to the condition of the patient 16. Furthermore, the second target value may change with the lapse of time according to the condition of the patient 16.

The second control section 154 controls a rotation speed of the second motor 136 based on the second target value calculated by the second setting section 152.

Here, setting processing in the second setting section 152 and control processing of the rotation speed of the second motor 136 in the second control section 154 will be described in detail.

Similarly to the first setting section 72, the second setting section 152 includes two maps (third map, fourth map). The third map is a map (not illustrated) indicating a relationship between the second flow rate and a second pressure (pressure of blood flowing through the second blood sending line 128). The second setting section 152 refers to the third map to convert the second target value into the second pressure.

The fourth map is a map of the pump characteristics of FIG. 3. The second setting section 152 refers to the fourth map to determine the rotation speed N according to the converted second pressure. The rotation speed N is a rotation speed of the second impeller 130. The second control section 154 controls the rotation speed of the second impeller 130 by controlling the rotation speed of the second motor 136 based on the determined rotation speed N. As a result, the second flow rate and the second pressure are controlled.

Note that the detection result of the second detection unit 95 is sequentially input to the second control unit 96. Therefore, the second setting section 152 and the second control section 154 may perform feedback control on the second flow rate based on the second target value and the detection result of the second detection unit 95.

The second display section 156 displays various display contents. The second communication section 158 transmits and receives signals or information to and from the first communication section 78 of the first control unit 24.

Incidentally, the condition of the patient 16 changes with the lapse of time. In a case where both the first blood circulation device 12 and the second blood circulation device 14 operate in the blood circulation system 10, it is necessary to appropriately adjust the flow rates of blood in the first blood circulation device 12 and the second blood circulation device 14 according to the condition of the patient 16. Therefore, in the blood circulation system 10 according to the present embodiment, the centrifugal pump of the other blood circulation device is controlled based on the detection result of the detection unit provided in one blood circulation device.

That is, as described above, the first communication section 78 and the second communication section 158 can mutually transmit and receive signals or information. Therefore, the communication section of one blood circulation device transmits the detection result of the detection unit provided in the one blood circulation device and the target flow rate to the communication section of the other blood circulation device. In the other blood circulation device, the centrifugal pump of the other blood circulation device is controlled based on the target flow rate and the detection result received by the communication section. In this case, the target flow rate is the sum of the target value (first target value) of the flow rate of the blood flowing from the first blood circulation device 12 to the ascending aorta 38 and the target value (second target value) of the flow rate of the blood flowing from the second blood circulation device 14 to the ascending aorta 38.

That is, in a case where both the first blood circulation device 12 and the second blood circulation device 14 operate, one blood circulation device functions as a main device for sending blood to the patient 16. The other blood circulation device functions as an auxiliary device for sending blood to the patient 16.

First, an operation in a case where the first blood circulation device 12 is an auxiliary device and the second blood circulation device 14 is a main device will be described.

First, the user inputs the target flow rate to the second input section 150 of the second control unit 96. Furthermore, the user inputs the second target value to the second input section 150. As a result, the second blood circulation device 14 sends blood to the patient 16 based on the second target value. In the present description, the second target value changes with the lapse of time according to the condition of the patient 16. Note that the target flow rate may change with the lapse of time according to the condition of the patient 16.

The second communication section 158 transmits the input target flow rate and the detection result (second flow rate) of the second detection unit 95 to the first communication section 78 of the first control unit 24.

The first communication section 78 outputs the target flow rate received from the second communication section 158 and the detection result of the second detection unit 95 to the first setting section 72. In a case where the detection result of the second detection unit 95 is the second flow rate, the first setting section 72 calculates the first flow rate (first target value) by subtracting the second flow rate from the target flow rate (first target value=target flow rate−second flow rate).

Next, the first setting section 72 refers to the first map and converts the calculated first target value into the first pressure. Next, the first setting section 72 refers to the second map to determine the rotation speed N of the first centrifugal pump 20 according to the converted first pressure.

The first control section 74 controls the rotation speed of the first impeller 56 by controlling the rotation speed of the first motor 62 based on the rotation speed N determined by the first setting section 72. As a result, since the first flow rate and the first pressure are controlled in consideration of the second flow rate or the second pressure, the pressure difference between the left ventricle 42 and the ascending aorta 38 can be appropriately adjusted.

It is desirable that the target flow rate and the detection result of the second detection unit 95 are sequentially transmitted from the second communication section 158 to the first communication section 78. As a result, in a case where the second target value changes from moment to moment according to the condition of the patient 16, the first setting section 72 can appropriately calculate the first target value according to the change in the second target value. As a result, the first control section 74 can appropriately control the rotation speed of the first centrifugal pump 20 based on the calculated first target value according to the condition of the patient 16.

Next, an operation in a case where the first blood circulation device 12 is a main device and the second blood circulation device 14 is an auxiliary device will be described.

The user inputs the target flow rate to the first input section 70 of the first control unit 24. Furthermore, the user inputs the first target value to the first input section 70. As a result, the first blood circulation device 12 sends blood to the patient 16 based on the first target value. In the present description, the first target value changes with the lapse of time according to the condition of the patient 16. Note that the target flow rate may change with the lapse of time according to the condition of the patient 16.

The first communication section 78 transmits the input target flow rate and the detection result (first flow rate) of the first detection unit 22 to the second communication section 158 of the second control unit 96.

The second communication section 158 outputs the target flow rate received from the first communication section 78 and the detection result of the first detection unit 22 to the second setting section 152. The second setting section 152 calculates the second flow rate (second target value) by subtracting the first flow rate from the target flow rate (second target value=target flow rate−first flow rate).

Next, the second setting section 152 refers to the third map and converts the calculated second target value into the second pressure. Next, the second setting section 152 refers to the fourth map to determine the rotation speed N of the second centrifugal pump 92 according to the converted second pressure.

The second control section 154 controls the rotation speed of the second impeller 130 by controlling the rotation speed of the second motor 136 based on the rotation speed N determined by the second setting section 152. As a result, the second flow rate and the second pressure are controlled in consideration of the first flow rate or the first pressure.

Preferably, the target flow rate and the detection result of the first detection unit 22 are sequentially transmitted from the first communication section 78 to the second communication section 158. As a result, in a case where the first target value changes from moment to moment according to the condition of the patient 16, the second setting section 152 can appropriately calculate the second target value according to the change in the first target value. As a result, the second control section 154 can appropriately control the rotation speed of the second centrifugal pump 92 based on the calculated second target value according to the condition of the patient 16.

Here, the above operation will be described by exemplifying specific numerical values. At the start of the operation of both the first blood circulation device 12 and the second blood circulation device 14, the first flow rate is set as follows. That is, in a case where the second blood circulation device 14 is the main device and the first blood circulation device 12 is the auxiliary device, the first flow rate is set as follows. When the target flow rate is 4.0 l/min and second detection unit 95 detects the second flow rate of 3.5 l/min, the first setting section 72 sets the first target value to 0.5 l/min (4.0−3.5=0.5).

Thereafter, when the recovery of the patient 16 progresses, the target flow rate is changed to 4.5 l/min, and the second detection unit 95 detects the second flow rate of 3.0 l/min, the first setting section 72 sets the first target value to 1.5 l/min (4.5−3.0=1.5). That is, in a case where the recovery of the patient 16 progresses, the flow rate of the blood in the second blood circulation device 14 is reduced, and the flow rate of the blood in the first blood circulation device 12 is increased.

As described above, since the centrifugal pump of the other blood circulation device can be controlled on the basis of the detection result of the detection unit provided in one blood circulation device, the rotation speeds of the first centrifugal pump 20 and the second centrifugal pump 92 can be simultaneously controlled. As a result, the first flow rate and the second flow rate can be appropriately controlled. For example, when the second flow rate changes, the pressure of the ascending aorta 38 connected to the femoral artery 126 changes. Therefore, by changing the first target value according to the change in the second flow rate, the rotation speed of the first centrifugal pump 20 changes. As a result, the first flow rate can be appropriately adjusted.

Furthermore, in the first blood circulation device 12, the cannula 34 is inserted into the subclavian artery 36. Therefore, in a case where the second blood circulation device 14 is removed from the patient 16, the feet of patient 16 can freely move. As a result, the patient 16 can perform rehabilitation early.

Moreover, in the above description, the case where the target flow rate is input to the input section of one blood circulation device (main device) has been described. In the present embodiment, the setting section of the other blood circulation device (auxiliary device) calculates the flow rate of the blood flowing through the other blood circulation device based on the target flow rate. Therefore, the user may input the target flow rate to the input section of the other blood circulation device.

Moreover, FIG. 1 illustrates a case where the first blood circulation device 12 and the second blood circulation device 14 include the control units 24 and 96, respectively. In some embodiments, the two control units 24 and 96 maybe collectively configured as one control unit.

Note that the present invention is not limited to the above-described embodiment, and various configurations can be taken without departing from the gist of the present invention.

### REFERENCE SIGNS LIST

10 BLOOD CIRCULATION SYSTEM
12 first blood circulation device
14 second blood circulation device
16 patient (living body)
20 first centrifugal pump
22 first detection unit (detection unit)
26 first blood removal vessel portion
28 first blood sending vessel portion
36 subclavian artery
38 ascending aorta
42 left ventricle
48 first inlet port
50 first outlet port
72 first setting section (flow rate calculation unit)
74 first control section
92 second centrifugal pump
95 second detection unit (detection unit)
98 blood removal cannula (second blood removal vessel portion)
110 blood sending cannula (second blood sending vessel portion)
112 femoral vein
116 second inlet port
120 second outlet port
126 femoral artery
152 second setting section (flow rate calculation unit)
154 second control section

What is claimed is:

1. A blood circulation system configured to remove blood from a living body of a patient and send the blood to the living body, the blood circulation system comprising:
a first blood circulation device;
a second blood circulation device; and
a detection unit;
wherein the first blood circulation device is comprised of:
a first blood removal vessel portion having a distal end inserted into a left ventricle of the living body via a subclavian artery of the living body;
a first blood sending vessel portion having a distal end inserted into an ascending aorta of the living body via the subclavian artery;
a first centrifugal pump that is disposed outside the living body, including a first inlet port and a first outlet port, wherein the first centrifugal pump removes blood from the left ventricle to the first inlet port via the first blood removal vessel portion, and wherein the first centrifugal pump sends blood from the first outlet port to the ascending aorta via the first blood sending vessel portion, the first inlet port being connected to a proximal end of the first blood removal vessel portion, the first outlet port being connected to a proximal end of the first blood sending vessel portion; and
a first control section that controls the first centrifugal pump;
wherein the second blood circulation device is comprised of:
a second blood removal vessel portion having a distal end inserted into a femoral vein of the living body;
a second blood sending vessel portion having a distal end inserted into a femoral artery of the living body;
a second centrifugal pump that is disposed outside the living body, including a second inlet port and a second outlet port, wherein the second centrifugal pump removes blood from the femoral vein to the second inlet port via the second blood removal vessel portion, and wherein the second centrifugal pump sends blood from the second outlet port to the femoral artery via the second blood sending vessel portion, the second inlet port being connected to a proximal end of the second blood removal vessel portion, the second outlet port being connected to a proximal end of the second blood sending vessel portion; and
a second control section that controls the second centrifugal pump;
wherein the detection unit is disposed outside the living body and detects a flow rate of blood flowing through the second blood sending vessel portion; and
wherein the first control section controls the first centrifugal pump based on a detection result of the detection unit.

2. The blood circulation system according to claim 1:
wherein the first blood circulation device comprises a flow rate calculation unit configured to calculate an auxiliary flow rate of blood targeted to flow into the first blood sending vessel portion based on (i) a target flow rate for the patient to flow in combination from the first blood circulation device and the second blood circulation device to the ascending aorta and (ii) the detection result of the detection unit; and
wherein the first control section controls a rotation speed of the first centrifugal pump based on the auxiliary flow rate calculated by the flow rate calculation unit.

3. The blood circulation system according to claim 2 further comprising a second input section in the second control section configured for a medical user to input the target flow rate for the patient to flow to the ascending aorta.

4. A blood circulation system configured to remove blood from a living body and send the blood to the living body, the blood circulation system comprising:
a first blood circulation device:
a second blood circulation device: and a detection unit;
wherein the first blood circulation device is comprised of:
a first blood removal vessel portion having a distal end inserted into a left ventricle of the living body via a subclavian artery of the living body;
a first blood sending vessel portion having a distal end inserted into an ascending aorta of the living body via the subclavian artery;
a first centrifugal pump that is disposed outside the living body, including a first inlet port and a first outlet port, wherein the first centrifugal pump removes blood from the left ventricle to the first inlet port via the first blood removal vessel portion, and wherein the first centrifugal pump sends blood from the first outlet port to the ascending aorta via the first blood sending vessel portion, the first inlet port being connected to a proximal end of the first blood removal vessel portion, the first outlet port being connected to a proximal end of the first blood sending vessel portion; and
a first control section that controls the first centrifugal pump;
wherein the detection unit is disposed outside the living body and detects a flow rate of blood flowing through the first blood sending vessel portion;
wherein the second blood circulation device is comprised of:
a second blood removal vessel portion having a distal end inserted into a femoral vein of the living body;
a second blood sending vessel portion having a distal end inserted into a femoral artery of the living body;
a second centrifugal pump that is disposed outside the living body, including a second inlet port and a second outlet port, wherein the second centrifugal pump removes blood from the femoral vein to the second inlet port via the second blood removal vessel portion, and wherein the second centrifugal pump sends blood from the second outlet port to the femoral artery via the second blood sending vessel portion, the second inlet port being connected to a proximal end of the second blood removal vessel portion, the second outlet port being connected to a proximal end of the second blood sending vessel portion;
a second control section that controls the second centrifugal pump; and
wherein the second control section controls the second centrifugal pump based on a detection result of the detection unit.

5. The blood circulation system according to claim 4:
wherein the second blood circulation device comprises a flow rate calculation unit configured to calculate an auxiliary flow rate of blood targeted to flow into the second blood sending vessel portion based on (i) a target flow rate flowing in combination from the first blood circulation device and the second blood circulation device and (ii) the detection result of the detection unit; and
wherein the second control section controls a rotation speed of the second centrifugal pump based on the auxiliary flow rate calculated by the flow rate calculation unit.

6. The blood circulation system according to claim 5 further comprising a first input section in the first control section configured for a medical user to input the target flow rate for the patient to flow to the ascending aorta.

7. A method of circulating blood of a living body of a patient using a first blood circulation device, a second blood circulation device, and a detection unit, the method comprising the steps of:
connecting the first blood circulation device to the patient so that a first centrifugal pump removes blood from a left ventricle of the patient and sends blood to an ascending aorta of the patient;
connecting the second blood circulation device to the patient so that a second centrifugal pump removes blood from a femoral vein of the patient and sends blood to a femoral artery of the patient;
setting a target flow rate for the patient to flow to the ascending aorta;
detecting a main flow rate of blood flowing through one of the first blood circulation device or the second blood circulation device to generate a detection result;
calculating an auxiliary flow rate of blood targeted to flow in the other one of the first blood circulation device or the second blood circulation device based on the target flow rate that was set and the detection result; and
controlling a rotation speed of the centrifugal pump in the other one of the first blood circulation device or the second blood circulation device according to the calculated auxiliary flow rate.

8. The method of claim 7 wherein the target flow rate for the patient to flow to the ascending aorta is set by a medical user, and wherein the auxiliary flow rate is communicated from the one of the first blood circulation device or the second blood circulation device to the other one of the first blood circulation device or the second blood circulation device.
